(19) United States
(12) Patent Application Publication
(10) Pub. No.: US 2023/0064630 A1
(43) Pub. Date: Mar. 2, 2023

(54) ALGAL BLOOM MEASURING SYSTEM USING IOT

(71) Applicant: Industry-Academic Cooperation Foundation, Dankook University, Yongin-si (KR)

(72) Inventor: Eun Ju LIM, Yongin-si (KR)

(73) Assignee: Industry-Academic Cooperation Foundation, Dankook University, Yongin-si (KR)

(21) Appl. No.: 17/473,966

(22) Filed: Sep. 13, 2021

(30) Foreign Application Priority Data
Aug. 25, 2021 (KR) 10-2021-0112047

(51) Int. Cl. G01N 33/00; G01N 27/12; G16Z 99/00; B63B 35/00

(52) U.S. Cl. CPC G01N 33/0098; G01N 27/12; G16Z 99/00; B63B 35/00; B63B 2035/006

(57) ABSTRACT

Disclosed is an algal bloom measuring system includes a body that floats on a water surface in a divided area, an algal bloom sensor unit disposed in the body and that generates an electric current through algae distributed in water, a controller that detects the electric current generated by the algal bloom sensor unit and determine whether algae are present, and a communication unit that transmits alga determination information of the controller to an outside.

### CROSS-REFERENCE TO RELATED APPLICATIONS

A claim for priority under 35 U.S.C. § 119 is made to Korean Patent Application No. 10-2021-0112047 filed on Aug. 25, 2021, in the Korean Intellectual Property Office, the entire contents of which are hereby incorporated by reference.

### BACKGROUND

Embodiments of the inventive concept described herein relate to an algal bloom measuring system using IoT.

In general, non-point source contaminants are also called nonspecific contaminants, surface contaminants, flow contaminants, or other water contaminants, and refer to contaminants that are discharged from a non-point contamination source that is a discharge source that does not specifically discharges water contaminants at a certain site such as a city, a road, an agricultural land, a mountain, or a construction site.

Although the point contaminants have specific discharge paths, the non-point contaminants are generated through unspecific discharge paths, such as city road surface drainages, or agricultural land drainages.

In particular, main causes of the water contaminations in branches of rivers or branches of small streams, such as a river, a reservoir, or a dam are indiscreet discharges of livestock sludge, factory wastewater, and domestic sewage, and contamination due to illegal discharges of factory wastewater and domestic sewage occupy 70% of a total water contaminants or more.

For a method for securing purified water by improving the problems of water contamination, technologies for measuring a quality of contaminated water in branches of rivers or small streams have been variously suggested.

However, the existing measurement technologies are limited to main streams as they include large-scale facilities, and thus their installation is limited.

Accordingly, it is difficult to recognize water quality states of various target areas in real time.

### SUMMARY

Embodiments of the inventive concept provide a real-time algal bloom measuring system based on IoT, which is configured to monitor an algal bloom state of a target area in real time.

The technical problems that are to be solved by the inventive concept are not limited to the above-mentioned ones, and the other technical problems that have not been mentioned will be clearly understood from the following description by an ordinary person in the art, to which the inventive concept pertains.

According to an embodiment, an algal bloom measuring system includes a body that floats on a water surface in a divided area, an algal bloom sensor unit disposed in the body and that generates an electric current through algae distributed in water, a controller that detects the electric current generated by the algal bloom sensor unit and determine whether algae are present, and a communication unit that transmits alga determination information of the controller to an outside.

Furthermore, the algal bloom sensor unit may be constituted by sequentially laminating a first electrode, a first hydrogel layer, a nafion film. a second hydrogel including potassium ferricyanide, and a second electrode, and the algal bloom sensor unit may generate an electric current when the algae in the water are introduced into the first hydrogel layer.

Furthermore, the controller may calculate an amount of algae distributed in the water by determining a magnitude of the electric current generated by the algal bloom sensor unit.

Furthermore, the algal bloom measuring system may further include an ultrasonic wave generating unit disposed in the body and that generates ultrasonic waves for removing the algae.

Furthermore, the algal bloom measuring system may further include a propulsion unit disposed in the body and that feeds the body, and a location sensor unit disposed in the body and that generates location information of the body.

Furthermore, the body may include at least one float part that generates buoyancy, and the algal bloom sensor unit may be disposed on a surface of the float part.

Furthermore, the float part may include a float member, at least one connection member coupled to an outer surface of the float member, and a net-shaped support frame disposed to be spaced apart from the outer surface of the float member through the connection member, and the algal bloom sensor unit may be disposed on a surface of the support frame.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is an exemplary view schematically illustrating an algal bloom measuring system according to an embodiment of the inventive concept;

FIG. 2 is an exemplary view schematically illustrating a configuration of an algal bloom measuring apparatus in an algal bloom measuring system according to an embodiment of the inventive concept;

FIG. 3 is a block diagram schematically illustrating a configuration of an algal bloom measuring apparatus in an algal bloom measuring system according to an embodiment of the inventive concept;

FIG. 4 is an exemplary view illustrating disposition of an algal bloom sensor unit in an algal bloom measuring system according to an embodiment of the inventive concept;

FIG. 5 is a cross-sectional view illustrating a configuration of an algal bloom sensor unit in an algal bloom measuring system according to an embodiment of the inventive concept; and

FIGS. 6 and 7 are exemplary views illustrating disposition of an algal bloom sensor unit in an algal bloom measuring system according to another embodiment of the inventive concept.

### DETAILED DESCRIPTION

Hereinafter, exemplary embodiments of the inventive concept will be described in more detail with reference to the accompanying drawings. The embodiments of the inventive may be modified in various forms, and the scope of the inventive concept should not be construed to be limited to the following embodiments. The embodiments of the inventive concept are provided to describe the inventive concept for an ordinary person skilled in the art more completely. Accordingly, the shapes of the components of the drawings are exaggerated to emphasize clearer description thereof.

The configurations of the inventive concept for clearly describing a solution for the problem that is to be solved by the inventive concept will be described in detail with reference to the accompanying drawings based on a preferred embodiment of the inventive concept, in which the same reference numerals are given for the same elements in denoting the reference numerals for the elements even though they are present in different drawings, and when a drawing has to be referenced for a description of the embodiment, the elements in another drawing also may be cited.

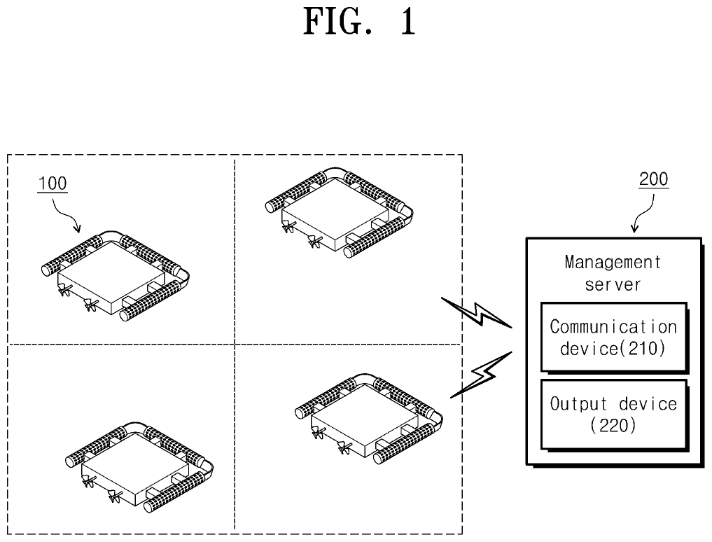

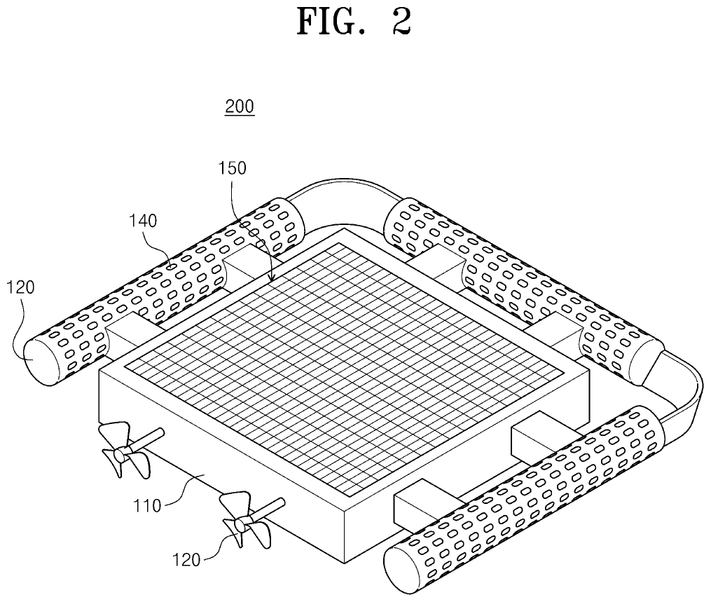

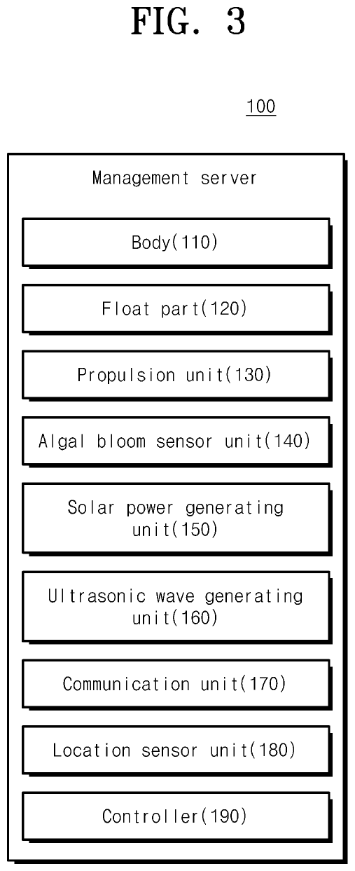

FIG. 1 is an exemplary view schematically illustrating an algal bloom measuring system according to an embodiment of the inventive concept. FIG. 2 is an exemplary view schematically illustrating a configuration of an algal bloom measuring apparatus in an algal bloom measuring system according to an embodiment of the inventive concept. FIG. 3 is a block diagram schematically illustrating a configuration of an algal bloom measuring apparatus in an algal bloom measuring system according to an embodiment of the inventive concept.

First, referring to FIG. 1, an algal bloom measuring system according to an embodiment of the inventive concept may include an algal bloom measuring apparatus 100 and a management server 200.

The algal bloom measuring apparatus 100 may float on a surface of water, such as a river or a sea, and be fixed, or may move to a required location and be disposed.

A plurality of algal bloom measuring apparatuses 100 may be disposed in divided areas, and may measure water quality states of the areas. Through this, each of the plurality of algal bloom measuring apparatuses 100 may evaluate the water qualities while moving in the corresponding divided area.

Meanwhile, the algal bloom measuring apparatus 100 and the management server 200 may perform wireless communication by using a communication unit.

Wireless communication refers to transmitting and receiving information, such as signals, symbols, images, and voices, through electric waves without using electric wires. Wireless communication technologies include wireless networks, wireless internets, wireless LANs, mobile communications, and near field communications.

That is, the algal bloom measuring apparatus 100 floats on a water surface of an ocean, and may communicate with an outside through a communication network (LoRa, LTE, 5G, and the like).

Here, one algal bloom measuring apparatus 100 may communicate with the management server 200, and may communicate with another algal bloom measuring apparatus 100 located in another area as well.

The algal bloom measuring apparatus 100 and the management server 200 may transmit and receive signals and information through a communication unit. That is, the algal bloom measuring apparatus 100 may transmit the water quality state and state information of the algal bloom measuring apparatus 100 to the management server 200, and the algal bloom measuring apparatus 100 may be driven according to a control signal of the management server 200.

The management server 200 collectively determines the water quality states of all the areas, in which the algal bloom measuring apparatuses 100 are distributed, based on various pieces of measurement elements provided by the algal bloom measuring apparatuses 100. That is, the management server 200, for example, may recognize whether algal bloom states of the target areas are severe or good, and the like.

Furthermore, the management server 200 may transmit various pieces of measurement information provided by the algal bloom measuring apparatuses 100 to a manager terminal, and may output them through an output unit that is embedded.

Referring to FIGS. 2 and 3, each of the algal bloom measuring apparatuses 100 may a body 110, a float part 120, a propulsion unit 130, an algal bloom sensor unit 140, a solar power generating unit 150, an ultrasonic wave generating unit 160, a communication unit 170, a location sensor unit 180, and a controller 190.

Meanwhile, although not described in detail, it is apparent that the algal bloom measuring apparatus 100 may include at least one sensor (not illustrated) of a weather cock, an anemometer, an eupatheoscope, a ceilometer, a visibility meter, a sea current direction meter, a speed meter, a wave-height meter, a wave recorder, or a rain gauge for measuring and recording at least one of a wind direction, a wind speed, an atmospheric humidity, an atmospheric pressure, an atmospheric temperature, a wave height, a visibility, a wave a wave height, a sea current speed, a sea current direction, or a precipitation of an area, in which the algal bloom measuring apparatus 100 is located.

The body 110 is a kind of a housing and includes an installation space, and may include float parts 120 fixed to at least opposite sides thereof and a portion of the body 110 may float on a water surface and be disposed. A gravitational force due to the weight of the body 110 and a buoyant force applied by the float part 120 are balanced, and the algal bloom measuring apparatus 100 float on the water surface while being balanced.

It is preferable that the body 110 is formed of a plastic material, such as PE, PA, or PP, which is corrosion-resistant, durable, and light-weighted.

Meanwhile, the body 110 is provided with an inner installation space, and it is preferable that the configurations are coupled to be separable for repair and replacement, and it is preferable that watertight members (not illustrated) are disposed in the separated areas to prevent introduction of seawater or freshwater.

Here, the float part 120 may mean a member that generates buoyancy, and may mean a float member provided to float on a water surface in a target area to measure a water quality of the target area including non-point source contaminants. Then, the non-point source contaminants may mean water pollutants generated in non-point contamination sources, such as marine product farming facilities, golf course facilities, agricultural lands, and product storage yards.

Unlike point source contaminants, such as domestic sewage, industrial wastewater, and livestock wastewater, which are generated at specific points, the non-point source contaminants may be generated through unspecific discharge paths, such as road surface drainages of cities or drainages of agricultural lands, and a buoy member that measures a water quality of a target area including the non-point source contaminants may be provided.

The target areas may include areas having deep water levels and narrow widths, such as branches of rivers or branches of small streams. That is, the embodiment may be applied to measure the water qualities of natural streams, braches of rivers, or branches of small streams.

Meanwhile, the body 110 has a sealed installation space in an interior thereof.

An anchor (not illustrated) is provided on one side of the body 110 and the anchor is fixed to a bottom of the target area, whereby the algal bloom measuring apparatus 100 is maintained in a state, in which it is stopped on a water surface of a target area that requires measurement of a water quality thereof.

Although not illustrated in detail, the anchor may be connected to one side of the body 110 through a wire, a winding unit for winding the wire, and a driving motor that rotates and drives the winding unit, and the driving motor may is driven in forward and reverse directions to perform a winding operation according to a control of the controller 190.

Meanwhile, because a single algal bloom measuring apparatus 100 may determine a central portion of a divided area based on a location sensor, such as a GPS, to measure a water quality in the allocated area, fixes the anchor to the central portion, and drives the propulsion unit 130 to be moved in a rotational direction about the anchor located at the central portion, a desired operation may be performed while it is maintained at its proper place in spite of waves.

A battery (not illustrated) is accommodated in an interior of the body 110, and the battery may supply electric power to various components provided in the algal bloom measuring apparatus 100. The battery stores electric energy supplied from the algal bloom sensor unit 140 and the solar power generating unit 150.

Meanwhile, the electric energy stored in the battery may be used as electric power for driving the algal bloom measuring apparatus 100, and the battery may include a secondary battery that may be charged and discharged. Here, it is preferable that the battery includes a lithium ion secondary battery that may have a high output. Meanwhile, the battery may include one or rechargeable battery cells, and a circuit module (not illustrated) that is electrically connected to electrode terminals of the battery cell to protect battery cells and measure states of charge (SOCs) of the battery cells.

It is apparent that the circuit module may measure the SOCs of the unit battery cells that constitute the battery, and it is possible to the charging states and the heat emission states of the battery cells.

Meanwhile, the controller 170 may monitor the SOC of the battery of the algal bloom measuring apparatus 100, may determine that the algal bloom measuring apparatus 100 is damaged due to a defect of the battery or an external impact when the SOC of the battery is less than a preset reference value, and may compulsorily move the algal bloom measuring apparatus 100 to a station on a seashore.

Here, the reference value for the SOC may be corrected according to a distance between the algal bloom measuring apparatus 100 and the station, and the reference values for the SOCs of the plurality of algal bloom measuring apparatuses 100 may be different based on location information.

Excessive electric power other than the driving electric power used for driving may be stored in the battery and be supplied to an external energy storage device (ESS) (not illustrated).

Meanwhile, regenerative energy generated through the algal bloom sensor unit 140 and the solar power generating unit 150 may be delivered to the battery to charge the battery, and the stored electric power may be sold to external electric power systems to create additional profits. That is, the algal bloom measuring system according to the inventive concept may allow energy harvesting by converting energy generated from a natural energy source into electric energy.

Here, the algal bloom measuring apparatus 100 may reach a station to charge the battery or deliver residual electric power, or an electric power feeding body (not illustrated) that stores and distributes electric power may approach the algal bloom measuring apparatus 100 for an electric power distribution procedure.

The propulsion unit 130 may be driven according to a control of the controller 190, and may include an electric propulsion body, such as a propeller, to move the algal bloom measuring apparatus 100 to a preset location.

The propulsion unit 130 may receive electric power from the battery, and may be rotated in a specific direction to generate a propulsion force.

Meanwhile, an end of a rotary shaft of the propeller may be connected to the body 110 to be rotatable, and through this, the propulsion unit 130 may determine a travel direction of the body 110.

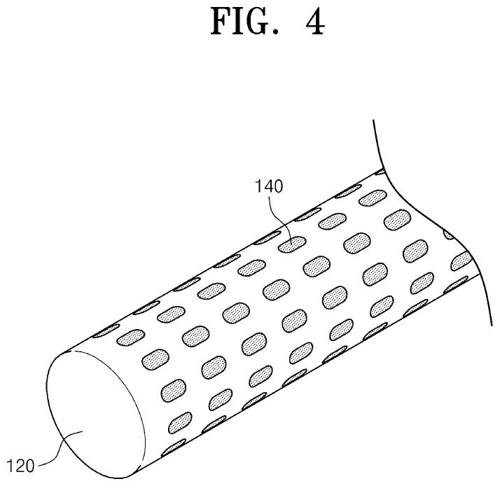

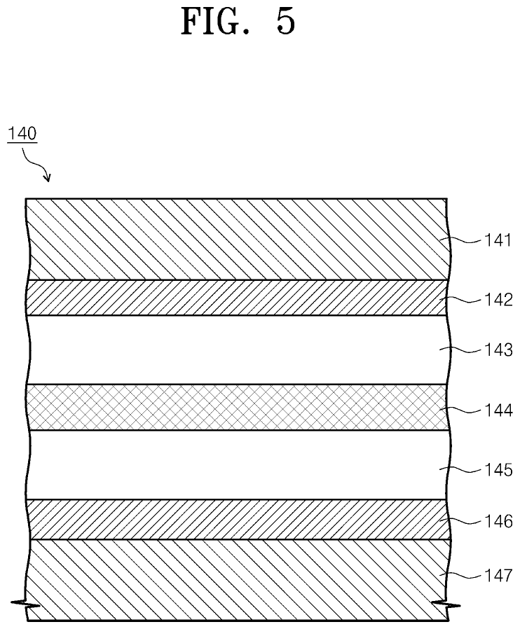

FIG. 4 is an exemplary view illustrating disposition of an algal bloom sensor unit in an algal bloom measuring system according to an embodiment of the inventive concept. FIG. 5 is a cross-sectional view illustrating a configuration of an algal bloom sensor unit in an algal bloom measuring system according to an embodiment of the inventive concept.

First, referring to FIGS. 2 and 4 again, a plurality of algal bloom sensor units 140 may be disposed along a surface of the float part 120.

Furthermore, although not illustrated in detail, the plurality of algal bloom sensor units 140 may be disposed along a bottom surface and a side surface of the float part 120. Through this, the algal bloom sensor unit 140 may have a large area in the single algal bloom measuring apparatus 100.

Meanwhile, according to the algal bloom sensor unit 140, it is preferable that the algal bloom sensor unit 140 is attached at a location of an outer peripheral surface of the float part 120, which is fully immersed in water when the body 110 floats on the water surface.

Furthermore, it is preferable that the algal bloom sensor unit 140 is selectively disposed at an upper portion and a side portion of the float part 120 such that input light is not blocked by the body 110.

Furthermore, it is preferable that the plurality of algal bloom sensor units 140 are disposed at an upper portion and a side portion of the float part 120 more densely.

Meanwhile, as the water surface is changed due to the wave height and the like, the algal bloom sensor unit 140 attached to the float part 120 may continuously contract the seawater or the freshwater.

Here, although not illustrated, the plurality of algal bloom sensor units 140 attached to the float part 120 may be electrically connected to each other, and currents generated by the algal bloom sensor units 140 may be stored in the battery.

Referring to FIG. 5, each of the algal bloom sensor units 140 includes a first protective film 141, a first ITO electrode 142, a first hydrogel layer 143, a nafion film 144, a second hydrogel layer 145, a second ITO electrode 146, and a second protective film 147.

A structure, in which the first ITO electrode 142, the first hydrogel layer 143, the nafion film 144, the second hydrogel layer 145, and the second ITO electrode 146 are sequentially laminated, may be provided between the first protective film 141 and the second protective film 147, and the second ITO electrode 146 may further include a graphene monolayer.

The second ITO electrode 146 may be a graphene electrode or an electrode including a graphene film. The graphene film itself may function as an electrode, or the second ITO electrode 146 including the graphene film may be used by coating graphene on a surface of the second ITO electrode.

Any transparent electrode may be applied instead of the ITO electrode.

Meanwhile, the term ‘hydrogel’ used in the inventive concept refers to a hydrophilic gel, in which a 3-dimensional network structure called aqua gel is formed, and represents an elasticity that is almost similar to a natural tissue due to the content of water. The hydrogel that may be included in the inventive concept may include one that may provide a survival environment of cells without limitation, and for example, may include a smart gel that detects a pH, a temperature, or a concentration of metabolite, silicon hydrogel, poly acryl amide hydrogel, agarose hydrogel, methyl cellulose hydrogel, polyvinyl alcohol hydrogel, sodium poly acrylate hydrogel, acrylate hydrogel, chondroitin hydrogel, glucosamine hydrogel, glycosaminoglycan hydrogel, fibrin hydrogel, fibrinogen hydrogel, thrombin hydrogel, hyaluronic acid hydrogel, collagen hydrogel, and the like, but the inventive concept is not limited thereto.

Meanwhile, when algal cells in the water are introduced into the first hydrogel layer 143, the algal bloom sensor unit 140 may generate electric currents.

Here, the algal cells in the water may be blue-green algae or green algae. Blue-green algae or green algae often discovered in rivers and seas may generate hydrogen ions with electrons through photosynthesis due to strong propagation potential, and thus the inventive concept may collect electrons and hydrogen ions from them so that it is suitable to determine whether algae are present in the water based on the generated electric currents.

Meanwhile, the nafion film 144 that separates electrodes is an ion conductive polymeric electrode film, and functions to deliver hydrogen ions between electrodes.

Furthermore, the second hydrogel layer 145 including potassium ferricyanide functions to collect hydrogen ions generated by the algal cells in the first hydrogel layer 143 when the hydrogen ions are diffused through the nafion film 144.

The solar power generating unit 150 may include a solar module that is disposed on the body 110 and generates electric currents through the input light. Although not illustrated, the solar power generating unit 150 may include a solar module and a driving unit that controls an angle of the solar module.

The ultrasonic wave generating unit 160 may be disposed in the body 110, and may be driven based on a control signal of the controller 190 or a control signal of the management server 200, and may remove algae in the corresponding area by generating ultrasonic waves through an ultrasonic vibrator.

Furthermore, the communication unit 140 for communicating with the management server 200 is installed in the body 110.

The communication unit 140 may include an input/output control device that is connected to a system through a transmission control device, and a remote terminal unit that refers to at least one input/output control device, and may communicate with the management server 200, and thus may transmit information sensed by the algal bloom measuring apparatus 100 to the management server 200 or receive a control signal from the management server 200 and transmit the control signal to the controller 190.

The location sensor unit 180 is installed in the body 110 to measure a location of the algal bloom measuring apparatus 100, and the location information of the algal bloom measuring apparatus 100 may be delivered to the controller 190 and may be delivered to the management server 200 through the communication unit 170.

The controller 190 is disposed in the body 110 to control various components. Here, the controller 190 may be implemented by a program mounted on a microcomputer or may be implemented on a substrate in a chip-on-board type through chip sets.

Meanwhile, the controller 190 may determine electric currents generated by the algal bloom sensor unit 140, and thus may determine that algae are generated in the corresponding area and determine an amount of generated algae based on an intensity of the electric currents.

As an example, the controller 190 may numericalize the current values generated per unit time by the algal bloom sensor unit 140, and may determine that a large amount of algae are generated in an area of a high numerical value and, to the contrary, may determine that a small amount of algae are generated in an area of a low numerical value.

Furthermore, as another example, the controller 190 may determine that the amount of the algae in the corresponding area is larger than a reference value when the value of the electric currents generated by the algal bloom sensor unit 140 is larger than a reference value, and determine that the amount of the algae in the corresponding area is smaller than the reference value when the value of the electric currents generated by the algal bloom sensor unit 140 is smaller than the reference value.

Referring to FIG. 4, at least one algal bloom sensor unit 140 is attached to the body 110 of the algal bloom measuring apparatus 100.

It is preferable that the algal bloom sensor unit 140 has a hollow cylindrical shape, and may be formed of transparent reinforced glass or plastic. The plurality of algal bloom sensor units 140 may be disposed on an inner surface 11 and an outer surface 14 of the cylindrical shape.

The management server 200 may communicate with the communication units 140 of the algal bloom measuring apparatuses 100 through a communication device 210, and may transmit a control signal of the algal bloom measuring apparatus 100 or receive algal bloom sensing information.

An output device 220 may be implemented by a terminal of a manager or a large-scale display device, and may output location information of the algal bloom measuring apparatuses 100, alga presence information at the corresponding location, and alga amount information.

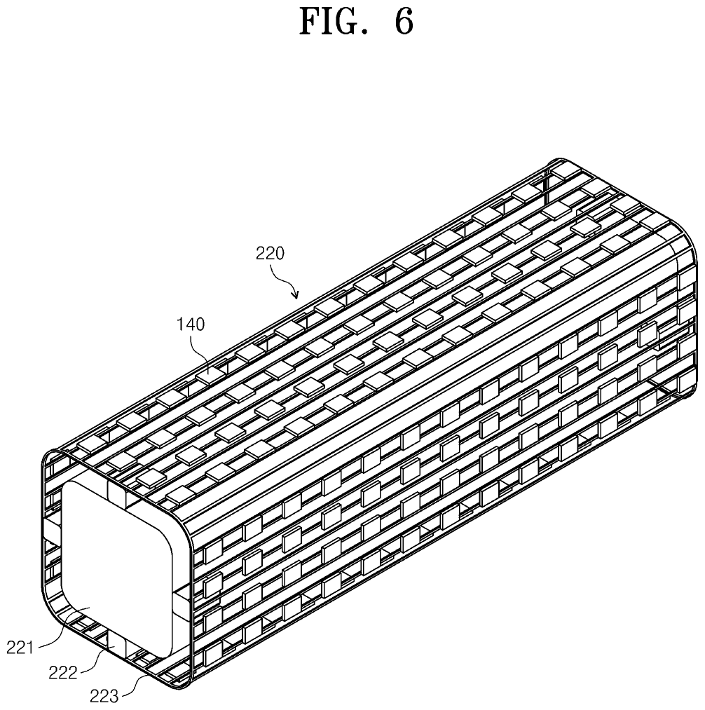

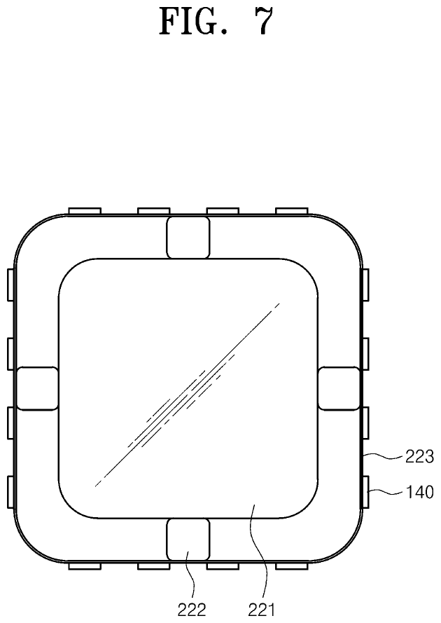

FIGS. 6 and 7 are exemplary views illustrating disposition of an algal bloom sensor unit in an algal bloom measuring system according to another embodiment of the inventive concept.

Referring to FIGS. 6 and 7, the algal bloom measuring system according to another embodiment of the inventive concept is the same as the algal bloom measuring system according to the embodiment of the inventive concept illustrated in FIG. 2 except for the float part 220, and thus only the float part 220 and the algal bloom sensor unit 140 disposed in the float part 220, which are distinguished from those of FIG. 2 will be described in detail; and the reference numerals of the repeated configurations will not be described in detail.

Referring to FIG. 2 together, the float part 220 of the algal bloom measuring system according to another embodiment of the inventive concept may be disposed in the same area, in which the float part 120 of the algal bloom measuring system 100 according to the embodiment of the inventive concept is disposed, and although not illustrated in detail in FIGS. 6 and 7, the float part 220 may be connected to the body 110.

Furthermore, it is apparent that the adjacent ones of the plurality of float parts 220 may be connected to each other.

The float part 220 may include a float member 221, a connection member 222, and a support frame 223.

The float member 221 may mean a member that generates buoyancy, and may mean a float member provided to float on a water surface in a target area to measure a water quality of the target area including non-point source contaminants.

Meanwhile, although the float member 221 and the support frame 223 have rectangular vessel shapes in FIGS. 6 and 7, the illustration is merely for detail illustration for convenience of description, and the inventive concept is not limited thereto.

Furthermore, the float member 221 and the support frame 223 have different sizes and the same shape, but may have different shapes.

One end of the connection member 223 may be coupled to and fixed to an outer surface of the float member 221, and may protrude from the outer surface of the float member 221 outwards.

A plurality of transverse ribs and longitudinal ribs of the support frame 223 may cross each other. Here, the support frame 223 may have a net shape having a plurality of holes on an outer surface and an inner surface thereof.

It is preferable that the support frame 223 may be connected to the float member 221 through the connection member 223, and the support frame 223 and the float member 221 are attached to each other.

Here, the support frame 223 is spaced apart from the float member 221, and a space between the outer surface of the float member 221 and the inner surface of the support frame 223 is opened so that the surrounding seawater or freshwater may be easily introduced and discharged.

The plurality of algal bloom sensor units 140 may be disposed along the inner surface or the outer surface of the support frame 223. Meanwhile, although not illustrated, the plurality of algal bloom sensor units 140 may be disposed on the inner surface and the outer surface of the support frame 223 to be zigzagged.

Furthermore, although not illustrated in detail, the plurality of algal bloom sensor units 140 may be disposed along a bottom surface and a side surface of the support frame 223. Through this, the algal bloom sensor unit 140 may have a large area in the single algal bloom measuring apparatus 100.

Meanwhile, it is preferable that the algal bloom sensor unit 140 is attached at a location of an outer peripheral surface of the support frame 223, which is fully immersed in water when the body 110 floats on the water surface.

Furthermore, it is preferable that the algal bloom sensor unit 140 is selectively disposed at an upper portion and a side portion of the float part 223 such that input light is not blocked by the body 110.

Furthermore, it is preferable that the plurality of algal bloom sensor units 140 are disposed at an upper portion and a side portion of the support frame 223 more densely.

Through this, the algal bloom sensor unit 140 may contact the seawater or the freshwater on a rear surface as well as a front surface thereof, and thus an algal bloom degree of the water may be measured more easily.

According to the embodiment of the inventive concept, it is possible to monitor an algal bloom state of a target area in real time.

The advantageous effects of the inventive concept are not limited to the above-mentioned ones, and the other advantageous effects will be clearly understood by an ordinary person skilled in the art to which the inventive concept pertains.

The above detailed description exemplifies the inventive concept. Furthermore, the above-mentioned contents describe the exemplary embodiment of the inventive concept, and the inventive concept may be used in various other combinations, changes, and environments. That is, the inventive concept can be modified and corrected without departing from the scope of the inventive concept that is disclosed in the specification, the equivalent scope to the written disclosures, and/or the technical or knowledge range of those skilled in the art. The written embodiment describes the best state for implementing the technical spirit of the inventive concept, and various changes required in the detailed application fields and purposes of the inventive concept can be made. Accordingly, the detailed description of the inventive concept is not intended to restrict the inventive concept in the disclosed embodiment state. Furthermore, it should be construed that the attached claims include other embodiments.

What is claimed is:

1. An algal bloom measuring system comprising:
a body configured to float on a water surface in a divided area;
an algal bloom sensor unit disposed in the body and configured to generate an electric current through algae distributed in water;
a controller configured to detect the electric current generated by the algal bloom sensor unit and determine whether algae are present; and
a communication unit configured to transmit alga determination information of the controller to an outside.

2. The algal bloom measuring system of claim 1, wherein the algal bloom sensor unit is constituted by sequentially laminating a first electrode, a first hydrogel layer, a nafion film, a second hydrogel including potassium ferricyanide, and a second electrode, and
wherein the algal bloom sensor unit generates an electric current when the algae in the water are introduced into the first hydrogel layer.

3. The algal bloom measuring system of claim 2, wherein the controller calculates an amount of algae distributed in the water by determining a magnitude of the electric current generated by the algal bloom sensor unit.

4. The algal bloom measuring system of claim 2, further comprising:
an ultrasonic wave generating unit disposed in the body and configured to generate ultrasonic waves for removing the algae.

5. The algal bloom measuring system of claim 2, further comprising:
a propulsion unit disposed in the body and configured to feed the body; and
a location sensor unit disposed in the body and configured to generate location information of the body.

6. The algal bloom measuring system of claim 2, wherein the body includes at least one float part configured to generate buoyancy, and wherein the algal bloom sensor unit is disposed on a surface of the float part.

7. The algal bloom measuring system of claim 6, wherein the float part includes:
a float member;
at least one connection member coupled to an outer surface of the float member; and
a net-shaped support frame disposed to be spaced apart from the outer surface of the float member through the connection member, and
wherein the algal bloom sensor unit is disposed on a surface of the support frame.
